(19) United States
(12) Patent Application Publication
(10) Pub. No.: US 2023/0313145 A1
(43) Pub. Date: Oct. 5, 2023

(54) METHOD FOR SCREENING IN VITRO POPULATION OF STEM CELL DERIVED BETA LIKE CELLS AND NOVEL MARKERS THEREOF

(71) Applicant: Novo Nordisk A/S, Bagsvaerd (DK)

(72) Inventors: Jeannette Schlichting Kirkegaard, Birkeroed (DK); Tess Tsai-Hsiu Lu, Taipei City (TW)

(21) Appl. No.: 18/022,538

(22) PCT Filed: Aug. 27, 2021
(86) PCT No.: PCT/EP2021/073782

§ 371 Date: Feb. 22, 2023

(30) Foreign Application Priority Data
Aug. 28, 2020 (EP) 20193349.6

(51) Int. Cl. C12N 5/071

(52) U.S. Cl. CPC C12N 5/0678

(57) ABSTRACT

The present invention relates to a method for screening for beta like cells in an in vitro cell population of pluripotent stem cell derived cells, wherein the method comprises a step of identifying the beta like cells expressing specific markers or combinations thereof for predicting the in vivo functionality of said cells prior to transplantation. The present invention also relates to an in vitro population of pluripotent stem cell derived beta like cells, wherein the beta like cell comprises one or more markers that is absent in native human beta cell or the expression level of said marker is different than in native human beta cells.

### TECHNICAL FIELD

The present invention relates to a method for screening for beta like cells in an in vitro population of pluripotent stem cell derived cells by identifying the beta like cells expressing one or more specific markers. The present invention also relates to in vitro population of pluripotent stem cell derived cells comprising beta like cells expressing specific markers, one or more markers absent in native human beta cells or having a different expression level than in native human beta cells.

### BACKGROUND

Patients with type 1 diabetes can be treated with transplantation of pancreatic islets from human donors and some patients achieve insulin independence. However, donor islets are scarce and of variable quality, hence, pluripotent stem cell derived beta like cells offer an attractive alternative to pancreatic islets.

During in vitro differentiation, human pluripotent stem cells are differentiated using a variety of chemical and biological factors to yield a heterogenous islet like cell clusters that comprise different endocrine cell types.

Classical beta cell markers include insulin (INS) and canonical transcription factors such as PDX1 and NKX6.1. Conventionally, these markers were used to identify stem cells derived beta like cells. Although all the stem cell derived beta like cells may appear phenotypically similar to native human beta cells by expression of the classical beta cell markers in vitro, they differ as not all the stem cell derived beta like cells express and secrete insulin after transplantation. Classical beta cell markers are insufficient to identify the stem cells derived beta like cells in vitro that maintain insulin expression and secretion after transplantation. Some of the classical beta cell markers such as PDX1 are even negatively correlated to in vivo function resulting in stem cell to beta like cell in vitro differentiation protocol optimisation towards undesired cell populations or selection of cell populations that may resemble the phenotype of native human beta cells before transplantation but change their phenotype after transplantation.

Identification of in vitro population of stem cell derived beta like cells that maintain insulin expression and secretion after transplantation is essential for the differentiated beta like cell product performance, assessment and production. Thus, there is a need to identify new markers that define the in vitro population of stem cell derived beta like cells that maintain insulin expression and secretion after transplantation.

### SUMMARY

In one aspect, the present invention relates to a method for screening for beta like cells in an in vitro population of pluripotent stem cell derived cells, wherein the method comprises the step of identifying the beta like cells expressing NKX6.1 in combination with one or more markers selected from ACVR1C, FREM2, CHRNA3, DCC, SOX11, MARCKSL1, BASP1, STARD10, AMBP, ST6GALNAC5, HMGCS1, ELAVL2, PCP4, PCDH7, NEFL, PLAGL1, EGFL7, RAD21, RTN1, PLXNA2, LBH, NEFM, SLC30A8, DLK1, MAFB and ISL1.

In one aspect, the present invention relates to an in vitro population of pluripotent stem cell derived cells, wherein at least 55% of the beta like cells comprise one or more markers selected from SOX11, FREM2, DCC, BASP1, CHRNA3, and ELALV2, wherein said marker is absent in native human beta cell.

In one aspect the present invention relates to an in vitro population of pluripotent stem cell derived cells, wherein at least 80% of the beta like cells comprise one or more markers selected from ACVR1C, MARCKSL1, STARD10, AMBP, ST6GALNAC5, and HMGCS1, wherein the expression level of said marker is at least about 1 average log fold change more than the expression of said marker in native human beta cell.

In one aspect, the present invention relates to an in vitro population of pluripotent stem cell derived cells comprising beta like cells for use in the treatment of type 1 diabetes.

In one aspect, the present invention relates to a cell composition, comprising an in vitro population of pluripotent stem cell derived cells comprising beta like cells as obtained by the present invention and a cell culture medium.

In one aspect, the present invention relates to an implantable device comprising an in vitro population of pluripotent stem cell derived cells comprising beta like cells obtained by the present invention.

In one aspect, the present invention relates to a method of treatment of type 1 diabetes, comprising administering to a person in need thereof, of an in vitro population of pluripotent stem cell derived cells comprising beta like cells obtained by the present invention.

In one aspect, the present invention relates to a cryopreserved in vitro population of pluripotent stem cell derived cells comprising beta like cells obtained by the present invention.

In one aspect, the present invention provides an in vitro population of pluripotent stem cells comprising beta like cells or insulin producing cells prior to transplantation.

The present invention may also solve further problems that will be apparent from the disclosure of the exemplary embodiments.

### BRIEF DESCRIPTION OF DRAWINGS

FIG. 1A is a graph showing T-distributed Stochastic Neighbor Embedding (tSNE) projection of all cells sampled from eight different (modified) protocols for obtaining stem cell derived cells, and from cadaveric human islet cells.

FIG. 1B is a graph showing tSNE projection of 26 cell clusters where each cluster consists of cells having similar gene expression profiles.

FIG. 2 is a graph showing stem cell derived cluster number on the X axis and beta cell score on the Y-axis. It shows that based on human beta cell gene module scoring of all cells stem cell derived beta like cell cluster numbers 14, 15 and 26 mostly resemble the native human beta cell cluster number 16.

FIG. 3 is a graph showing native islet subtypes on the X axis and beta cell score on the Y axis. All cells scoring high on the beta cell gene set were classified as native human beta cells.

FIG. 4 is a graph showing gene expression profiles unique to an in vitro population of stem cell derived beta like cells. Native human beta cells (cluster 16) were compared to stem cell derived beta like cells (clusters 14, 15 and 26). Median UPT, which is a measure of RNA quality, for native human beta cells equals to 0. Median UPT for stem cell derived beta like cells was greater than for native human beta cells. The plots show the average expression difference between primary or native human beta cells and stem cell derived beta like cells using a box plot and the expression in the individual cells using a single dot. Below each column is indicated a percentage of cells that show no expression.

FIG. 5 is a figure showing stem cell derived cells before and after transplantation in vivo. It shows that some stem cell derived cells do not remain insulin positive after in vivo exposure. NKX6.1, Insulin and Glucagon staining of stem cell derived cells before and after transplantation shows that although the cells look similar to native human beta cells before transplantation they differ significantly in insulin expression and secretion after transplantation.

FIG. 6 is a graph showing percentage of cells in clusters 14, 15 and 26 on the X axis and average human C-peptide 8 weeks after transplantation on Y-axis. It shows a positive correlation between stem cell derived beta like cells and human C-peptide secreted 8 week after in vivo exposure. C-peptide levels measured in SCID-beige animals transplanted with cells generated using different (modified) differentiation protocols show positive correlation to the accumulated size of cluster 14, 15, and 26.

FIG. 7 are graphs showing that classical beta cell markers NKX6.1, SYT13, NKX2.2, PDX1, PDX1+/NKX6.1+, PAX4 have a negative correlation to average human C-peptide 8 weeks after transplantation in SCID mice.

FIG. 8 a) upper panel is a graph showing that SLC30A8 is enriched in the stem cell derived beta like cell population.

FIG. 8 a) lower panel is a graph showing broad expression of PDX1.

FIG. 8 b) upper panel is a graph showing that SLC30A8 is positively correlated with average human c-peptide after 8 weeks after transplantation in SCID mice.

FIG. 8 b) lower panel is a graph showing that PDX1 is negatively correlated to average human c-peptide 8 weeks after transplantation in SCID mice.

FIG. 9 a) shows that ACVR1C is not expressed in native human beta cells.

FIG. 9 b) shows that ACVR1C marks a subset of cells within the stem cell derived cell culture.

FIG. 9 c) is a graph showing that ACVR1C expression positively correlates to in vivo function.

FIG. 10 a) upper panel is a tSNE plot showing high expression of PCDH7 in cluster 14, 15 and 26.

FIG. 10 a) lower panel is a tSNE plot showing high expression of PCP4 in cluster 14, 15 and 26.

FIG. 10 b) upper panel is a graph showing a positive correlation of PCDH7 with average human C-peptide after 8 weeks of in vivo exposure in SCID-beige mice.

FIG. 10 b) lower panel is a graph showing a positive correlation of PCP4 with average human C-peptide after 8 weeks of in vivo exposure in SCID-beige mice.

FIG. 11 a) is a tSNE plot showing G6PC2 expression.

FIG. 11 b) is a graph showing that the bulk expression of G6PC2 mRNA measured by nanostring correlates to in vivo insulin secretion in the form of circulating average human C-peptide 8 weeks after transplantation in SCID mice.

FIG. 11 c) is a graph showing that the bulk expression of G6PC2 mRNA measured by nanostring correlates to in vitro insulin secretion in the form of human C-peptide secreted during a glucose exendin4 and IBMX/forskolin challenge.

### DESCRIPTION

The present invention relates to a method for screening for beta like cells in an in vitro population of pluripotent stem cell derived cells comprising identifying beta like cells expressing one or more specific markers. The markers and the method of the present invention allow the identification of beta like cells, prior to transplantation, that will maintain insulin expression and secretion after transplantation. This is possible because the stem cell derived beta like cells identified according to the present invention do not change their phenotype after transplantation unlike the stem cell derived beta like cells identified based on the expression of classical beta cell markers.

Further, the present invention provides specific novel markers or application of classical beta cell markers in identification of stem cell derived beta like cells that maintain insulin expression and secretion after transplantation.

Furthermore, the present invention allows identification of beta like cells that maintain insulin expression and secretion after transplantation by detection of markers that are either absent in native human beta cells or have higher expression level in in vitro population of stem cell derived beta like cells than in native human beta cells.

In one aspect according to the present invention, the marker genes and combinations thereof are for use in protein based assays such as but not limited to proteomics, flow cytometry, immunohistochemistry or RNA based assays such as but not limited to RNA sequencing, qPCR probe based detection and other analytical or screening methods to identify or purify the stem cell derived beta cells that maintain insulin expression and secretion after transplantation for assessing differentiation efficiency, quality and performance.

In one aspect, the present invention relates to a method for screening for beta like cells in an in vitro population of pluripotent stem cell derived cells, wherein the method comprises the step of identifying the beta like cells expressing NKX6.1 in combination with one or more markers selected from ACVR1C, FREM2, CHRNA3, DCC, SOX11, MARCKSL1, BASP1, STARD10, AMBP, ST6GALNAC5, HMGCS1, ELAVL2, PCP4, PCDH7, NEFL, PLAGL1, EGFL7, RAD21, RTN1, PLXNA2, LBH, NEFM, SLC30A8, DLK1, MAFB and ISL1.

In one aspect, the present invention relates to a method for screening for beta like cells in an in vitro population of pluripotent stem cell derived cells, wherein the method comprises the step of identifying the beta like cells expressing NKX6.1 in combination with one or more markers selected from FREM2, CHRNA3, DCC, SOX11, MARCKSL1, BASP1, STARD10, AMBP, ST6GALNAC5, HMGCS1, ELAVL2, PCP4, PCDH7, NEFL, PLAGL1, EGFL7, RAD21, RTN1, PLXNA2, LBH, NEFM, DLK1 and MAFB.

In one aspect, the present invention relates to a method for screening for beta like cells in an in vitro population of pluripotent stem cell derived cells, wherein the method comprises the step of identifying the beta like cells expressing NKX6.1 in combination with one or more markers selected from CHRNA3, SOX11, MARCKSL1, BASP1, STARD10, AMBP, ST6GALNAC5, HMGCS1, ELAVL2, PCP4, PCDH7, NEFL, PLAGL1, EGFL7, RAD21, RTN1, PLXNA2, LBH, NEFM, DLK1 and MAFB.

In one aspect, the present invention relates to a method for screening for beta like cells in an in vitro population of pluripotent stem cell derived cells, wherein the method comprises the step of identifying the beta like cells expressing NKX6.1 in combination with one or more markers selected from CHRNA3, SOX11, MARCKSL1, BASP1, STARD10, AMBP, ST6GALNAC5, HMGCS1, ELAVL2, PLAGL1, EGFL7, RAD21, RTN1, LBH, and DLK1.

In one embodiment the present invention relates to a method for screening for beta like cells in an in vitro population of pluripotent stem cell derived cells, wherein the method comprises the step of identifying the beta like cells expressing NKX6.1 in combination with ACVR1C.

In one embodiment the present invention relates to a method for screening for beta like cells in an in vitro population of pluripotent stem cell derived cells, wherein the method comprises the step of identifying the beta like cells expressing NKX6.1 in combination with FREM2.

In one embodiment the present invention relates to a method for screening for beta like cells in an in vitro population of pluripotent stem cell derived cells, wherein the method comprises the step of identifying the beta like cells expressing NKX6.1 in combination with CHRNA3.

In one embodiment the present invention relates to a method for screening for beta like cells in an in vitro population of pluripotent stem cell derived cells, wherein the method comprises the step of identifying the beta like cells expressing NKX6.1 in combination with DCC.

In one embodiment the present invention relates to a method for screening for beta like cells in an in vitro population of pluripotent stem cell derived cells, wherein the method comprises the step of identifying the beta like cells expressing NKX6.1 in combination with SOX11.

In one embodiment the present invention relates a method for screening for beta like cells in an in vitro population of pluripotent stem cell derived cells, wherein the method comprises the step of identifying the beta like cells expressing NKX6.1 in combination with MARCKSL1.

In one embodiment the present invention relates to a method for screening for beta like cells in an in vitro population of pluripotent stem cell derived cells, wherein the method comprises the step of identifying the beta like cells expressing NKX6.1 in combination with BASP1.

In one embodiment the present invention relates to a method for screening for beta like cells in an in vitro population of pluripotent stem cell derived cells, wherein the method comprises the step of identifying the beta like cells expressing NKX6.1 in combination with STARD10.

In one embodiment the present invention relates to a method for screening for beta like cells in an in vitro population of pluripotent stem cell derived cells, wherein the method comprises the step of identifying the beta like cells expressing NKX6.1 in combination with AMBP.

In one embodiment the present invention relates to a method for screening for beta like cells in an in vitro population of pluripotent stem cell derived cells, wherein the method comprises the step of identifying the beta like cells expressing NKX6.1 in combination with ST6GALNAC5.

In one embodiment the present invention relates to a method for screening for beta like cells in an in vitro population of pluripotent stem cell derived cells, wherein the method comprises the step of identifying the beta like cells expressing NKX6.1 in combination with HMGCS1.

In one embodiment the present invention relates to a method for screening for beta like cells in an in vitro population of pluripotent stem cell derived cells, wherein the method comprises the step of identifying the beta like cells expressing NKX6.1 in combination with ELAVL2.

In one embodiment the present invention relates to a method for screening for beta like cells in an in vitro population of pluripotent stem cell derived cells, wherein the method comprises the step of identifying the beta like cells expressing NKX6.1 in combination with PCP4.

In one embodiment the present invention relates to a method for screening for beta like cells in an in vitro population of pluripotent stem cell derived cells, wherein the method comprises the step of identifying the beta like cells expressing NKX6.1 in combination with PCDH7.

In one embodiment the present invention relates to a method for screening for beta like cells in an in vitro population of pluripotent stem cell derived cells, wherein the method comprises the step of identifying the beta like cells expressing NKX6.1 in combination with NEFL.

In one embodiment the present invention relates to a method for screening for beta like cells in an in vitro population of pluripotent stem cell derived cells, wherein the method comprises the step of identifying the beta like cells expressing NKX6.1 in combination with PLAGL1.

In one embodiment the present invention relates to a method for screening for beta like cells in an in vitro population of pluripotent stem cell derived cells, wherein the method comprises the step of identifying the beta like cells expressing NKX6.1 in combination with EGFL7.

In one embodiment the present invention relates to a method for screening for beta like cells in an in vitro population of pluripotent stem cell derived cells, wherein the method comprises the step of identifying the beta like cells expressing NKX6.1 in combination with RAD21.

In one embodiment the present invention relates to a method for screening for beta like cells in an in vitro population of pluripotent stem cell derived cells, wherein the method comprises the step of identifying the beta like cells expressing NKX6.1 in combination with RTN1.

In one embodiment the present invention relates to a method for screening for beta like cells in an in vitro population of pluripotent stem cell derived cells, wherein the method comprises the step of identifying the beta like cells expressing NKX6.1 in combination with PLXNA2.

In one embodiment the present invention relates to a method for screening for beta like cells in an in vitro population of pluripotent stem cell derived cells, wherein the method comprises the step of identifying the beta like cells expressing NKX6.1 in combination with LBH.

In one embodiment the present invention relates to a method for screening for beta like cells in an in vitro population of pluripotent stem cell derived cells, wherein the method comprises the step of identifying the beta like cells expressing NKX6.1 in combination with NEFM.

In one embodiment the present invention relates a method for screening for beta like cells in an in vitro population of pluripotent stem cell derived cells, wherein the method comprises the step of identifying the beta like cells expressing NKX6.1 in combination with SLC30A8.

In one embodiment the present invention relates to a method for screening for beta like cells in an in vitro population of pluripotent stem cell derived cells, wherein the method comprises the step of identifying the beta like cells expressing NKX6.1 in combination with DLK1.

In one embodiment the present invention relates to a method for screening for beta like cells in an in vitro population of pluripotent stem cell derived cells, wherein the method comprises the step of identifying the beta like cells expressing NKX6.1 in combination with MAFB.

In one embodiment the present invention relates to a method for screening for beta like cells in an in vitro population of pluripotent stem cell derived cells, wherein the method comprises the step of identifying the beta like cells expressing NKX6.1 in combination with ISL1.

Methods for assessing expression of protein and nucleic acid markers in in vitro cell populations include qualitative reverse transcriptase polymearse chain reaction (RT-PCR), Northern blots, in situ hybridization (see, e.g. Current Protocols in Molecular Biology (Asubel et al., eds. 2001 supplement), and immunoassays such as immunohistochemical analysis of sectioned material, western blotting, and for markers that are accessible in intact cells, flow cytometry analysis (FACS) (see, e.g., Harlow and Lane, Using Antibodies: A Laboratory Manual, New York: Cold Spring Harbor Laboratory Press (1998)).

In one aspect the present invention relates an in vitro population of pluripotent stem cell derived cells, wherein the population comprises at least 15% beta like cells expressing NKX6.1 in combination with one or more markers selected from ACVR1C, FREM2, CHRNA3, DCC, SOX11, MARCKSL1, BASP1, STARD10, AMBP, ST6GALNAC5, HMGCS1, ELAVL2, PCP4, PCDH7, NEFL, PLAGL1, EGFL7, RAD21, RTN1, PLXNA2, LBH, NEFM, SLC30A8, DLK1, MAFB and ISL1.

In one aspect, the present invention relates to an in vitro population of pluripotent stem cell derived cells, wherein the population comprises at least 20%, 30%, 40%, 50%, 60%, 70%, 80%, or 90% beta like cells expressing NKX6.1 in combination with one or more markers selected from ACVR1C, FREM2, CHRNA3, DCC, SOX11, MARCKSL1, BASP1, STARD10, AMBP, ST6GALNAC5, HMGCS1, ELAVL2, PCP4, PCDH7, NEFL, PLAGL1, EGFL7, RAD21, RTN1, PLXNA2, LBH, NEFM, SLC30A8, DLK1, MAFB, and ISL1.

In one aspect the present invention relates to an in vitro population of pluripotent stem cell derived cells, wherein the population comprises at least 15% beta like cells expressing NKX6.1 in combination with one or more markers selected from ACVR1C, FREM2, CHRNA3, DCC, SOX11, MARCKSL1, BASP1, STARD10, AMBP, ST6GALNAC5, HMGCS1, ELAVL2, PCP4, PCDH7, NEFL, PLAGL1, EGFL7, RAD21, RTN1, PLXNA2, LBH, NEFM, SLC30A8, DLK1, MAFB, and ISL1, and wherein said beta like cells maintain insulin expression and secretion after transplantation.

In one aspect, the present invention relates to an in vitro population of pluripotent stem cell derived cells, wherein the population comprises at least 20%, 30%, 40%, 50%, 60%, 70%, 80%, or 90% beta like cells expressing NKX6.1 in combination with one or more markers selected from ACVR1C, FREM2, CHRNA3, DCC, SOX11, MARCKSL1, BASP1, STARD10, AMBP, ST6GALNAC5, HMGCS1, ELAVL2, PCP4, PCDH7, NEFL, PLAGL1, EGFL7, RAD21, RTN1, PLXNA2, LBH, NEFM, SLC30A8, DLK1, MAFB, and ISL1, and wherein said beta like cells maintain insulin expression and secretion after transplantation.

In one aspect, the present invention relates to an in vitro population of pluripotent stem cell derived cells, wherein the population comprises at least 15% beta like cells expressing NKX6.1 in combination with one or more markers selected from FREM2, CHRNA3, DCC, SOX11, MARCKSL1, BASP1, STARD10, AMBP, ST6GALNAC5, HMGCS1, ELAVL2, PCP4, PCDH7, NEFL, PLAGL1, EGFL7, RAD21, RTN1, PLXNA2, LBH, NEFM, DLK1 and MAFB.

In one aspect, the present invention relates to an in vitro population of pluripotent stem cell derived cells, wherein the population comprises at least 15% beta like cells expressing NKX6.1 in combination with one or more markers selected from FREM2, CHRNA3, DCC, SOX11, MARCKSL1, BASP1, STARD10, AMBP, ST6GALNAC5, HMGCS1, ELAVL2, PCP4, PCDH7, NEFL, PLAGL1, EGFL7, RAD21, RTN1, PLXNA2, LBH, NEFM, DLK1 and MAFB, and wherein said beta like cells maintain insulin expression and secretion after transplantation.

In one aspect, the present invention relates to an in vitro population of pluripotent stem cell derived cells, wherein the population comprises at least 15% beta like cells expressing NKX6.1 in combination with one or more markers selected from CHRNA3, SOX11, MARCKSL1, BASP1, STARD10, AMBP, ST6GALNAC5, HMGCS1, ELAVL2, PCP4, PCDH7, NEFL, PLAGL1, EGFL7, RAD21, RTN1, PLXNA2, LBH, NEFM, DLK1 and MAFB.

In one aspect, the present invention relates to an in vitro population of pluripotent stem cell derived cells, wherein the population comprises at least 15% beta like cells expressing NKX6.1 in combination with one or more markers selected from CHRNA3, SOX11, MARCKSL1, BASP1, STARD10, AMBP, ST6GALNAC5, HMGCS1, ELAVL2, PCP4, PCDH7, NEFL, PLAGL1, EGFL7, RAD21, RTN1, PLXNA2, LBH, NEFM, DLK1 and MAFB, and wherein said beta like cells maintain insulin expression and secretion after transplantation.

In one aspect, the present invention relates to an in vitro population of pluripotent stem cell derived cells, wherein the population comprises at least 15% beta like cells expressing NKX6.1 in combination with one or more markers selected from CHRNA3, SOX11, MARCKSL1, BASP1, STARD10, AMBP, ST6GALNAC5, HMGCS1, ELAVL2, PLAGL1, EGFL7, RAD21, RTN1, LBH, and DLK1.

In one aspect, the present invention relates to an in vitro population of pluripotent stem cell derived cells, wherein the population comprises at least 15% beta like cells expressing NKX6.1 in combination with one or more markers selected from CHRNA3, SOX11, MARCKSL1, BASP1, STARD10, AMBP, ST6GALNAC5, HMGCS1, ELAVL2, PLAGL1, EGFL7, RAD21, RTN1, LBH, and DLK1, and wherein said beta like cells maintain insulin expression and secretion after transplantation.

In one embodiment the present invention relates to an in vitro population of pluripotent stem cell derived cells, wherein the population comprises at least 15% beta like cells expressing NKX6.1 in combination with ACVR1C.

In one embodiment the present invention relates to an in vitro population of pluripotent stem cell derived cells, wherein the population comprises at least 15% beta like cells expressing NKX6.1 in combination with FREM2.

In one embodiment the present invention relates to an in vitro population of pluripotent stem cell derived cells, wherein the population comprising at least 15% beta like cells expressing NKX6.1 in combination with CHRNA3.

In one embodiment the present invention relates to an in vitro population of pluripotent stem cell derived cells, wherein the population comprises at least 15% beta like cells expressing NKX6.1 in combination with DCC.

In one embodiment the present invention relates to an in vitro population of pluripotent stem cell derived cells, wherein the population comprises at least 15% beta like cells expressing NKX6.1 in combination with SOX11.

In one embodiment the present invention relates to an in vitro population of pluripotent stem cell derived cells, wherein the population comprises at least 15% beta like cells expressing NKX6.1 in combination with MARCKSL1.

In one embodiment the present invention relates to an in vitro population of pluripotent stem cell derived cells, wherein the population comprising at least 15% beta like cells expressing NKX6.1 in combination with BASP1.

In one embodiment the present invention relates to an in vitro population of pluripotent stem cell derived cells, wherein the population comprising at least 15% beta like cells expressing NKX6.1 in combination with STARD10.

In one embodiment the present invention relates to an in vitro population of pluripotent stem cell derived cells, wherein the population comprising at least 15% beta like cells expressing NKX6.1 in combination with AMBP.

In one embodiment the present invention relates to an in vitro population of pluripotent stem cell derived cells, wherein the population comprising at least 15% beta like cells expressing NKX6.1 in combination with ST6GALNAC5.

In one embodiment the present invention relates to an in vitro population of pluripotent stem cell derived cells, wherein the population comprising at least 15% beta like cells expressing NKX6.1 in combination with HMGCS1.

In one embodiment the present invention relates to an in vitro population of pluripotent stem cell derived cells, wherein the population comprising at least 15% beta like cells expressing NKX6.1 in combination with ELAVL2.

In one embodiment the present invention relates to an in vitro population of pluripotent stem cell derived cells, wherein the population comprising at least 15% beta like cells expressing NKX6.1 in combination with PCP4.

In one embodiment the present invention relates to an in vitro population of pluripotent stem cell derived cells, wherein the population comprising at least 15% beta like cells expressing NKX6.1 in combination with PCDH7.

In one embodiment the present invention relates to an in vitro population of pluripotent stem cell derived cells, wherein the population comprising at least 15% beta like cells expressing NKX6.1 in combination with NEFL.

In one embodiment the present invention relates to an in vitro population of pluripotent stem cell derived cells, wherein the population comprising at least 15% beta like cells expressing NKX6.1 in combination with PLAGL1.

In one embodiment the present invention relates to an in vitro population of pluripotent stem cell derived cells, wherein the population comprising at least 15% beta like cells expressing NKX6.1 in combination with EGFL7.

In one embodiment the present invention relates to an in vitro population of pluripotent stem cell derived cells, wherein the population comprising at least 15% beta like cells expressing NKX6.1 in combination with RAD21.

In one embodiment the present invention relates to an in vitro population of pluripotent stem cell derived cells, wherein the population comprising at least 15% beta like cells expressing NKX6.1 in combination with RTN1.

In one embodiment the present invention relates to an in vitro population of pluripotent stem cell derived cells, wherein the population comprising at least 15% beta like cells expressing NKX6.1 in combination with PLXNA2.

In one embodiment the present invention relates to an in vitro population of pluripotent stem cell derived cells, wherein the population comprising at least 15% beta like cells expressing NKX6.1 in combination with LBH.

In one embodiment the present invention relates to an in vitro population of pluripotent stem cell derived cells, wherein the population comprising at least 15% beta like cells expressing NKX6.1 in combination with NEFM.

In one embodiment the present invention relates to an in vitro population of pluripotent stem cell derived cells, wherein the population comprising at least 15% beta like cells expressing NKX6.1 in combination with SLC30A8.

In one embodiment the present invention relates to an in vitro population of pluripotent stem cell derived cells, wherein the population comprising at least 15% beta like cells expressing NKX6.1 in combination with DLK1.

In one embodiment the present invention relates to an in vitro population of pluripotent stem cell derived cells, wherein the population comprising at least 15% beta like cells expressing NKX6.1 in combination with MAFB.

In one embodiment the present invention relates to an in vitro population of pluripotent stem cell derived cells, wherein the population comprising at least 15% beta like cells expressing NKX6.1 in combination with ISL1.

In one aspect, the present invention relates to an in vitro population of pluripotent stem cell derived cells, wherein at least 55% of the beta like cells comprise one or more markers selected from SOX11, FREM2, DCC, BASP1, CHRNA3, and ELALV2, wherein said marker is absent in native human beta cell.

In one embodiment the present invention relates an in vitro population of pluripotent stem cell derived cells, wherein at least 85% of the beta like cells comprise SOX11 wherein said marker is absent in native human beta cell.

In one embodiment the present invention relates an in vitro population of pluripotent stem cell derived cells, wherein at least 75% of the beta like cells comprise FREM2 wherein said marker is absent in native human beta cell.

In one embodiment the present invention relates an in vitro population of pluripotent stem cell derived cells, wherein at least 85% of the beta like cells comprise DCC wherein said marker is absent in native human beta cell.

In one embodiment the present invention relates an in vitro population of pluripotent stem cell derived cells, wherein at least 75% of the beta like cells comprise BASP1 wherein said marker is absent in native human beta cell.

In one embodiment the present invention relates an in vitro population of pluripotent stem cell derived cells, wherein at least 80% of the beta like cells comprise CHRNA3 wherein said marker is absent in native human beta cell.

In one embodiment the present invention relates an in vitro population of pluripotent stem cell derived cells, wherein at least 55% of the beta like cells comprise ELALV2, wherein said marker is absent in native human beta cell.

In one aspect the present invention relates to an in vitro population of pluripotent stem cell derived cells, wherein at least 80% of the beta like cells comprise one or more markers selected from ACVR1C, MARCKSL1, STARD10, AMBP, ST6GALNAC5, and HMGCS1, wherein the expression level of said marker is at least about 1 average log fold change more than the expression of said marker in native human beta cell.

In one embodiment average log fold change equals to average log fold change between cell cluster of interest and all other cell clusters.

In one embodiment the present invention relates to an in vitro population of pluripotent stem cell derived cells, wherein at least 95% of the beta like cells comprise ACVR1C wherein the expression level of said marker is at least 2 average log fold change more than the expression of said marker in native human beta cell.

In one embodiment the present invention relates to an in vitro population of pluripotent stem cell derived cells, wherein at least 95% of the beta like cells comprise MARCKSL1 wherein the expression level of said marker is at least about 2 average log fold change more than the expression of said marker in native human beta cell.

In one embodiment the present invention relates to an in vitro population of pluripotent stem cell derived cells, wherein at least 95% of the beta like cells comprise STARD10 wherein the expression level of said marker is at least 1 average log fold change more than the expression of said marker in native human beta cell.

In one embodiment the present invention relates to an in vitro population of pluripotent stem cell derived cells, wherein at least 90% of the beta like cells comprise AMBP wherein the expression level of said marker is at least 2 average log fold change more than the expression of said marker in native human beta cell.

In one embodiment the present invention relates to an in vitro population of pluripotent stem cell derived cells, wherein at least 85% of the beta like cells comprise ST6GALNAC5 wherein the expression level of said marker is at least about 1 average log fold change more than the expression of said marker in native human beta cell.

In one embodiment the present invention relates to an in vitro population of pluripotent stem cell derived cells, wherein at least 80% of the beta like cells comprise HMGCS1 wherein the expression level of said marker is at least about 1 average log fold change more than the expression of said marker in native human beta cell.

In one aspect the present invention relates to an in vitro population of pluripotent stem cell derived cells, wherein at least 80% of the beta like cells comprise one or more markers selected from ACVR1C, MARCKSL1, STARD10, AMBP, ST6GALNAC5, and HMGCS1, wherein the expression level of said marker is at least 10%, 20%, 30%, 40%, 50%, 60%, 70%, 80% or 90% more than the expression of said marker in native human beta cell.

In one embodiment the present invention relates to an in vitro population of pluripotent stem cell derived cells comprising beta like cells that maintain insulin expression and secretion after transplantation.

In one embodiment the present invention relates to an in vitro population of pluripotent stem cell derived cells comprising beta like cells, wherein the cell population exhibit a glucose-stimulated insulin secretion (GSIS) response after transplantation.

In one embodiment the response is an in vitro GSIS response. In one embodiment the response is an in vivo GSIS response. In one embodiment, the GSIS response resembles the response of a native human beta cell.

In one embodiment the present invention relates to an in vitro population of pluripotent stem cell derived cells comprising beta like cells, wherein one or more markers exhibit a positive correlation with average plasma c-peptide levels after transplantation.

In one embodiment the present invention relates to an in vitro population of pluripotent stem cell derived cells comprising beta like cells, wherein the beta like cells exhibit a lowering of blood glucose after transplantation.

In one embodiment the present invention relates to an in vitro population of pluripotent stem cell derived cells comprising beta like cells, wherein the beta like cells are glucose responsive.

In one embodiment the present invention relates to in vitro population of stem cell derived cells comprising beta like cells, wherein beta like cells are glucose responsive after transplantation.

In one embodiment the present invention relates to the in vitro population of stem cell derived cells comprising beta like cells, wherein the beta like cells exhibits enhanced C-peptide levels after transplantation.

In one embodiment the present invention relates to the in vitro population of stem cell derived cells comprising beta like cells, wherein the beta like cells exhibit improved insulin levels after transplantation.

In one embodiment according to the present invention the in vitro population of stem cell derived cells comprising beta like cells, wherein the beta like cells exhibit a lowering of blood glucose after transplantation.

In one aspect, the present invention relates to an in vitro cell culture or composition, comprising a cell population of pluripotent stem cell derived cells wherein at least 15% beta like cells express NKX6.1 in combination with one or more markers selected from ACVR1C, FREM2, CHRNA3, DCC, SOX11, MARCKSL1, BASP1, STARD10, AMBP, ST6GALNAC5, HMGCS1, ELAVL2, PCP4, PCDH7, NEFL, PLAGL1, EGFL7, RAD21, RTN1, PLXNA2, LBH, NEFM, SLC30A8, DLK1, MAFB, and ISL1 and a cell culture medium.

In one aspect, the present invention relates to an in vitro cell culture or composition, comprising a cell population of pluripotent stem cell derived cells, wherein at least 55% of the beta like cells comprise one or more markers selected from SOX11, FREM2, DCC, BASP1, CHRNA3, and ELALV2 and a cell culture medium, wherein said marker is absent in native human beta cell.

In one aspect, the present invention relates to an in vitro cell culture or composition, comprising a cell population of pluripotent stem cell derived cells, wherein at least 80% of the beta like cells comprise one or more markers selected from ACVR1C, MARCKSL1, STARD10, AMBP, ST6GALNAC5, and HMGCS1 and a cell culture medium, wherein the expression level of said marker is at least 1 average log fold change more than the expression of said marker in native human beta cell.

In one embodiment, the cells, stem cells, stem cell derived cells and/or stem cell beta like cells of the present invention are human cells.

In one embodiment, the stem cell is an embryonic stem cell or induced pluripotent stem cell.

In one aspect, the present invention relates to an implantable device comprising an in vitro population of stem cell derived cells wherein at least 15% beta like cells express NKX6.1 in combination with one or more markers selected from ACVR1C, FREM2, CHRNA3, DCC, SOX11, MARCKSL1, BASP1, STARD10, AMBP, ST6GALNAC5, HMGCS1, ELAVL2, PCP4, PCDH7, NEFL, PLAGL1, EGFL7, RAD21, RTN1, PLXNA2, LBH, NEFM, SLC30A8, DLK1, MAFB, and ISL1.

In one aspect, the present invention relates to an implantable device comprising an in vitro population of stem cell derived cells wherein at least 55% of the beta like cells comprise one or more markers selected from SOX11, FREM2, DCC, BASP1, CHRNA3, and ELALV2, wherein said marker is absent in native human beta cell.

In one aspect, the present invention relates to an implantable device comprising an in vitro population of stem cell derived cells wherein at least 80% of the beta like cells comprise one or more markers selected from ACVR1C, MARCKSL1, STARD10, AMBP, ST6GALNAC5, and HMGCS1, wherein the expression level of said marker is at least 1 average log fold change more than the expression of said marker in native human beta cell.

In one aspect, the present invention relates to a method of treatment of type 1 diabetes, wherein the method comprises administering to a person in need thereof, of an in vitro population of stem cell derived cells wherein at least 15% beta like cells express NKX6.1 in combination with one or more markers selected from ACVR1C, FREM2, CHRNA3, DCC, SOX11, MARCKSL1, BASP1, STARD10, AMBP, ST6GALNAC5, HMGCS1, ELAVL2, PCP4, PCDH7, NEFL, PLAGL1, EGFL7, RAD21, RTN1, PLXNA2, LBH, NEFM, SLC30A8, DLK1, MAFB, and ISL1.

In one aspect, the present invention relates to a method of treatment of type 1 diabetes, wherein the method comprises administering to a person in need thereof, of an in vitro population of stem cell derived cells wherein at least 55% of the beta like cells comprise one or more markers selected from SOX11, FREM2, DCC, BASP1, CHRNA3, and ELALV2, wherein said marker is absent in native human beta cell.

In one aspect, the present invention relates to a method of treatment of type 1 diabetes, wherein the method comprises administering to a person in need thereof, of an in vitro population of stem cell derived cells wherein at least 80% of the beta like cells comprise one or more markers selected from ACVR1C, MARCKSL1, STARD10, AMBP, ST6GALNAC5 and HMGCS1, wherein the expression level of said marker is at least 1 average log fold change more than the expression of said marker in native human beta cell.

In one aspect, the present invention relates to a cryopreserved population of stem cell derived cells wherein at least 15% of the beta like cells express NKX6.1 in combination with one or more markers selected from ACVR1C, FREM2, CHRNA3, DCC, SOX11, MARCKSL1, BASP1, STARD10, AMBP, ST6GALNAC5, HMGCS1, ELAVL2, PCP4, PCDH7, NEFL, PLAGL1, EGFL7, RAD21, RTN1, PLXNA2, LBH, NEFM, SLC30A8, DLK1, MAFB, and ISL1.

In one aspect, the present invention relates to a cryopreserved population of stem cell derived cells wherein at least 55% of the beta like cells comprise one or more markers selected from SOX11, FREM2, DCC, BASP1, CHRNA3, and ELALV2, wherein said marker is absent in native human beta cell.

In one aspect, the present invention relates to a cryopreserved population of stem cell derived cells wherein at least 80% of the beta like cells comprise one or more markers selected from ACVR1C, MARCKSL1, STARD10, AMBP, ST6GALNAC5, and HMGCS1, wherein the expression level of said marker is at least 1 average log fold change more than the expression of said marker in native human beta cell.

### Definitions

Unless otherwise stated, all technical and scientific terms used herein have the same meaning as commonly understood by one of ordinary skill in the art to which this invention belongs. The practice of the present invention employs, unless otherwise indicated, conventional methods of chemistry, biochemistry, biophysics, molecular biology, cell biology, genetics, immunology and pharmacology, known to those skilled in the art.

It is noted that all headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Throughout this application the terms “method” and “protocol” when referring to processes for differentiating cells may be used interchangeably.

As used herein, “and/or” refers to and encompasses any and all possible combinations of one or more of the associated listed items, as well as the lack of combinations when interpreted in the alternative (“or”). Moreover, the present invention also contemplates that in some embodiments of the invention, any feature or combination of features set forth herein can be excluded or omitted

As used herein “a” means “one or more”.

As used herein the term “about” means plus or minus 10%, such as plus or minus, 5% unless indicated otherwise.

As used herein, the term “cell population” refers to a defined group of cells, which may be in vitro or in vivo. In a preferred embodiment, the cell population according to the present invention is an in vitro cell population.

Stem cells Stem cells are undifferentiated cells defined by their ability at the single cell level to both self-renew and differentiate to produce progeny cells, including self-renewing progenitors, non-renewing progenitors, and terminally differentiated cells. Stem cells are also characterized by their ability to differentiate in vitro into functional cells of various cell lineages from multiple germ layers (endoderm, mesoderm and ectoderm), as well as to give rise to tissues of multiple germ layers following transplantation and to contribute substantially to most, if not all, tissues following injection into blastocysts.

Stem cells are classified by their developmental potential as: (1) totipotent, meaning able to give rise to all embryonic and extraembryonic cell types; (2) pluripotent, meaning able to give rise to all embryonic cell types; (3) multi-potent, meaning able to give rise to a subset of cell lineages, but all within a particular tissue, organ, or physiological system (for example, hematopoietic stem cells (HSC) can produce progeny that include HSC (self-renewal), blood cell restricted oligopotent progenitors and all cell types and elements (e.g., platelets) that are normal components of the blood); (4) oligopotent, meaning able to give rise to a more restricted subset of cell lineages than multi-potent stem cells; and (5) unipotent, meaning able to give rise to a single cell lineage (e.g., spermatogenic stem cells).

Human Pluripotent Stem Cells

As used herein, “human pluripotent stem cells” (hPSC) refers to cells that may be derived from any human source and that are capable, under appropriate conditions, of producing progeny of different cell types that are derivatives of all the 3 germinal layers (endoderm, mesoderm, and ectoderm). hPSC may have the ability to form a teratoma in 8-12 week old SCID mice and/or the ability to form identifiable cells of all three germ layers in tissue culture. Included in the definition of human pluripotent stem cells are human embryonic stem cells (hESCs) (see, e.g., Thomson et al. (1998), Heins et al. (2004), as well as induced pluripotent stem cells (iPSCs) (see, e.g. Yu et al. (2007); Takahashi et al. (2007)). The various methods and other embodiments described herein may require or utilise hPSC from a variety of sources. For example, hPSC suitable for use may be obtained from developing embryos. Additionally, or alternatively, suitable hPSC may be obtained from established cell lines and/or human induced pluripotent stem (hiPS) cells.

ES cell lines can also be derived from single blastomeres without the destruction of ex utero embryos and without affecting the clinical outcome (Chung et al. (2006) and Klimanskaya et al. (2006)).

Blastocyst-Derived Stem Cell

As used herein, the term “blastocyst-derived stem cell” is denoted BS cell, and the human form is termed “hBS cells”. In literature the cells are often referred to as embryonic stem cells, and more specifically human embryonic stem cells (hESC). The pluripotent stem cells used in the present invention can thus be embryonic stem cells prepared from blastocysts, as described in e.g. WO 03/055992 and WO 2007/042225, or be commercially available hBS cells or cell lines. However, it is further envisaged that any human pluripotent stem cell can be used in the present invention, including differentiated adult cells which are reprogrammed to pluripotent cells by e.g. treating adult cells with certain transcription factors, such as OCT4, SOX2, NANOG, and LIN28 as disclosed in Yu, et al. (2007); Takahashi et al. (2007) and Yu et al. (2009).

Human Embryonic Stem Cells

As used herein, the term “Human embryonic stem cells” or “hESCs” refer to stem cells derived from the inner cell mass of a human embryo. Human Embryonic Stem cells are capable of self-renewing indefinitely in an undifferentiated state. Furthermore, Human Embryonic Stem Cells are pluripotent, meaning they are able to differentiate into all derivatives of the three primary germ layers: ectoderm, endoderm and mesoderm.

Human Induced Pluripotent Stem Cells

As used herein, the term “Human induced pluripotent stem cells” or “hIPSCs” cells refer to stem cells generated by reprogramming somatic cells back into an embryonic-like pluripotent state. Induced Pluripotent Stem Cells are capable of self-renewing indefinitely in an undifferentiated state. Induced Pluripotent Stem Cells are also able differentiate into all derivatives of the three primary germ layers: ectoderm, endoderm and mesoderm.

Differentiation

As used herein “differentiate” or “differentiation” refers to a process where cells progress from an undifferentiated state to a differentiated state, or from a differentiated state to a more differentiated state. Cells can be in a differentiated state, but not be fully differentiated into a specific cell type.

The term “differentiation factor” refers to a compound added to pancreatic cells to enhance their differentiation into endocrine cells where endocrine cells also contain insulin producing beta cells.

In one embodiment, differentiation of the cells comprises culturing the cells in a medium comprising one or more differentiation factors.

Definitive Endoderm Cells (DE Cells)

Definitive endoderm cells are characterised by expression of the marker SOX17. Further markers of DE are FOXA2 and CXCR4.

“SOX17” (SRY-box 17) as used herein is a member of the SOX (SRY-related HMG-box) family of transcription factors involved in the regulation of embryonic development and in the determination of the cell fate.

“FOXA2” (forkhead box A2) as used herein is a member of the forkhead class of DNA-binding proteins.

“CXCR4” (C-X-C motif chemokine receptor 4) as used herein is a CXC chemokine receptor specific for stromal cell-derived factor-1.

Non-limiting examples of DE inducing protocols is the conventional D'Amour protocol (Novocell, Nature Biotec 2006, 2008) and the protocol described in WO2012/175633 (which is incorporated herein by reference in its entirety).

Pancreatic Endoderm Cells (PE Cells)

Pancreatic endoderm cells are characterised by expression of markers at least 5% NKX6.1+/PDX1+ double positive. Further markers of PE are PTF1A and CPA1.

“PDX1” as used herein, refers to a homeodomain transcription factor implicated in pancreas development.

“NKX6.1” as used herein is a member of the NKX transcription factor family.

“PTF1A” as used herein is a protein that is a component of the pancreas transcription factor 1 complex (PTF1) and is known to have a role in mammalian pancreatic development.

“CPA1” as used herein is a member of the carboxypeptidase A family of zinc metalloproteases. This enzyme is produced in the pancreas.

The protocol described in WO2014/033322 is incorporated herein by reference in its entirety.

Endocrine Progenitor Cells (EP Cells)

Endocrine progenitor cells are characterised by expression of markers NGN3, NeuroD and NKX2.2, hallmarks for EP cells committed to an endocrine cell fate.

“NGN3” as used herein, is a member of the neurogenin family of basic loop-helix-loop transcription factors.

“NKX2.2” and “NKX6.1” as used herein are members of the NKX transcription factor family.

“NeuroD” as used herein is a member of the NeuroD family of basic helix-loop-helix (bHLH) transcription factors.

The protocol described in WO2015/028614 is incorporated herein by reference in its entirety.

Native Human Beta Cells

As used herein, the term “native human beta cell” or “primary beta cells” refers to cell producing insulin in response to glucose or secretagogues in the human body.

Stem Cell Derived Cells

As used herein the term “Stem cell derived cells” or “pluripotent stem cell derived cells” refers to cells that are obtained by differentiation of pluripotent stem cells that either have the potential of further differentiation or are terminally differentiated cells but may not necessarily demonstrate insulin secretion after transplantation.

Stem Cell Derived Beta Cells

As used herein, the term “Beta like cells” or “Stem cell derived beta cell” or “SC-8 cell” or “Stem cell derived beta like cells” or “Stem cell derived beta cells in vitro” refers to cells that are derived from stem cells and express at least one marker of a native human beta cell such as PDX1, NKX6.1, INS, demonstrates an in vitro and/or in vivo insulin secretion in response to glucose that resembles the response of a native human beta cell. The protocol described in WO2017/144695 is incorporated herein by reference in its entirety.

Screening

As used herein, the term “screening”, refers to detecting, identifying, purifying, selecting or characterising a cell or a cell population on the basis of presence of one or more cells having a certain phenotype of genotype. The phenotype or genotype may be established based on the expression of markers. In one embodiment, identifying refers to classifying a cell according to the expression level of a marker characterizing the cell. In a preferred embodiment, the screening according to the present invention is carried out in vitro.

Expression Level

As used herein, the term “expression level” refers to the degree of gene expression and/or gene product activity in a cell. Expression level can be determined in arbitrary absolute units or normalized units (relative to known expression levels of a control reference).

Markers

As used herein, the term “marker” refers to a naturally occurring identifiable expression made by a cell which can be correlated with certain properties of the cell and serves to identify, predict or characterise a cell or cell population. A markers may be referred to by gene. A marker may be in the form of mRNA or protein for e.g. protein on the cell surface.

As used herein, the term “expression” in reference to a marker refers to the lack or presence in the cell of a molecule, which can be detected. In an embodiment, the expressed molecule is mRNA or a protein. The expression of the marker may be detected at any suitable level, such as at mRNA or protein level. A person skilled in the art will readily appreciate that a cell can be defined by the positive or negative expression of a marker, i.e. the properties and state of a cell may equally be correlated based on the expression of a certain marker as well as the lack thereof. When referring to specific markers the presence or lack of expression may be denoted with + (plus) or − (minus) signs, respectively.
“ACVR1C” as used herein is activin A receptor type 10.
“AMBP” as used herein is alpha-1-microglobulin/bikunin precursor.
“BASP1” as used herein is a brain abundant membrane attached signal protein 1.
“CHRNA3” as used herein is cholinergic receptor nicotinic alpha 3 subunit.
“DCC” as used herein is a gene that encodes netrin 1 receptor.
“DLK1” as used herein is a delta like non-canonical notch ligand.
“EGFLT7” as used herein is a EGF like domain multiple 7.
“ELAVL2” as used herein is Embryonic Lethal, Abnormal Vision, Drosophila like 2
“FREM2” as used herein is FRAS1 related extracellular matrix 2.
“G6PC2” as used herein is glucose-6-phosphatase catalytic subunit 2.
“HMGCS1” as used herein is 3-hydroxy-3-methylglutaryl-CoA synthase 1.
“ISL1” as used herein is ISL LIM homeobox 1.
“LBH” as used herein is LBH regulator of WNT signaling pathway.
“MAFB” as used herein is MAF bZIP transcription factor B.
“MARCKSL1” as used herein is MARCKS like 1.
“NEFL” as used herein is neurofilament light.
“NEFM” as used herein is neurofilament medium.
“PCDH7” as used herein is protocadherin 7.
“PCP4” as used herein is Purkinje cell protein 4.
“PLAGL1” as used herein is PLAG1 like zinc finger 1.
“PLXNA2” as used herein is plexin A2.
“RAD21” as used herein is RAD21 cohesin complex component.
“RTN1” as used herein is reticulon 1.
“SLC30A8” as used herein is solute carrier family 30 member 8.
“SOX11” as used herein is SRY-box transcription factor 11.
“STARD10” as used herein is StAR related lipid transfer domain containing 10.
“ST6GALNAC5” as used herein is ST6 N-acetylgalactosaminide alpha-2,6-sialyltransferase 5.

Insulin Expression or Production

Insulin expression or production is the ability of a cell to synthesize insulin protein and/or insulin RNA. In a preferred embodiment, the beta like cells of the present invention maintain insulin expression after transplantation.

Insulin Secretion

Insulin secretion is the ability of the cells to release insulin. In a preferred embodiment, the beta like cells of the present invention maintain insulin secretion after transplantation.

Culture Medium/Composition

A solid, liquid or semi-solid designed to support the growth of cells. Different types of commercial media are used for growing different types of cells. In a preferred embodiment, the cell culture or composition according to the present invention is an in vitro cell culture or composition.

Unless otherwise indicated in the specification, terms presented in singular form also include the plural situation.

The invention is further described by the following non-limiting embodiments:

1. A method for screening for beta like cells in an in vitro population of pluripotent stem cell derived cells, wherein the method comprises the step of identifying the beta like cells expressing NKX6.1 in combination with one or more markers selected from ACVR1C, FREM2, CHRNA3, DCC, SOX11, MARCKSL1, BASP1, STARD10, AMBP, ST6GALNAC5, HMGCS1, ELAVL2, PCP4, PCDH7, NEFL, PLAGL1, EGFL7, RAD21, RTN1, PLXNA2, LBH, NEFM, SLC30A8, DLK1, MAFB, and ISL1.

2. The method according to embodiment 1, wherein the beta like cells are screened by RNAseq.

3. The method according to any one of the preceding embodiments, wherein the beta like cells are screened using qPCR, nested PCR, ddPCR, or a combination thereof.

4. An in vitro population of pluripotent stem cell derived cells, wherein the population comprises at least 15% beta like cells expressing NKX6.1 in combination with one or more markers selected from ACVR1C, FREM2, CHRNA3, DCC, SOX11, MARCKSL1, BASP1, STARD10, AMBP, ST6GALNAC5, HMGCS1, ELAVL2, PCP4, PCDH7, NEFL, PLAGL1, EGFL7, RAD21, RTN1, PLXNA2, LBH, NEFM, SLC30A8, DLK1, MAFB, and ISL1.

5. An in vitro population of pluripotent stem cell derived cells according to embodiment 4, wherein the population comprises at least 15% beta like cells expressing NKX6.1 in combination with one or more markers selected from ACVR1C, FREM2, CHRNA3, DCC, SOX11, MARCKSL1, BASP1, STARD10, AMBP, ST6GALNAC5, HMGCS1, ELAVL2, PCP4, PCDH7, NEFL, PLAGL1, EGFL7, RAD21, RTN1, PLXNA2, LBH, NEFM, SLC30A8, DLK1, MAFB, and ISL1, and wherein said beta like cells maintain insulin expression and secretion after transplantation.

6. An in vitro population of pluripotent stem cell derived cells, according to embodiment 4 or 5, wherein at least 55% of the beta like cells comprise one or more markers selected from SOX11, FREM2, DCC, BASP1, CHRNA3, and ELALV2, wherein said marker is absent in native human beta cell.

7. The in vitro population of pluripotent stem cell derived cells according to any of the embodiments 4 to 6, wherein at least 85% of the beta like cells comprise SOX11, wherein said marker is absent in native human beta cell.

8. The in vitro population of pluripotent stem cell derived cells according to any of the embodiments 4 to 6, wherein at least 75% of the beta like cells comprise FREM2, wherein said marker is absent in native human beta cell.

9. The in vitro population of pluripotent stem cell derived cells according to any of the embodiments 4 to 6, wherein at least 85% of the beta like cells comprise DCC, wherein said marker is absent in native human beta cell.

10. The in vitro population of pluripotent stem cell derived cells according to any of the embodiments 4 to 6, wherein at least 75% of the beta like cells comprise BASP1, wherein said marker is absent in native human beta cell.

11. The in vitro population of pluripotent stem cell derived cells according to any of the embodiments 4 to 6, wherein at least 80% of the beta like cells comprise CHRNA3, wherein said marker is absent in native human beta cell.

12. The in vitro population of pluripotent stem cell derived cells according to embodiments 4 to 6, wherein at least 55% of the beta like cells comprise ELALV2, wherein said marker is absent in native human beta cell.

13. The in vitro population of pluripotent stem cell derived cells according to any of the embodiments 4 to 9, wherein at least 75% of the beta like cells comprise SOX11, FREM2, DCC, wherein said marker is absent in native human beta cell.

14. The in vitro population of pluripotent stem cell derived cells according to any of the embodiments 10 to 12, wherein at least 55% of the beta like cells comprise BASP1, CHRNA3, ELAVL2.

15. An in vitro population pluripotent stem cell derived cells according to any one of the embodiments 4 or 5, wherein at least 80% of the beta like cells comprise one or more markers selected from ACVR1C, MARCKSL1, STARD10, AMBP, ST6GALNAC5, and HMGCS1, wherein the expression level of said marker is at least about 1 average log fold change more than the expression of said marker in native human beta cell.

16. An in vitro population pluripotent stem cell derived cells according to embodiments 15, wherein at least 90% of the beta like cells comprise one or more markers selected from ACVR1C, MARCKSL1, STARD10, AMBP, wherein the expression level of said marker is at least about 1 average log fold change more than the expression of said marker in native human beta cell.

17. The in vitro population pluripotent stem cell derived cells, according to any of the embodiments 15 or 16, wherein at least 95% of the beta like cells comprise ACVR1C, wherein the expression level of said marker is at least 2 average log fold change more than the expression of said marker in native human beta cell.

18. The in vitro population pluripotent stem cell derived cells, according to any of the embodiment 15 or 16, wherein at least 95% of the beta like cells comprise MARCKSL1, wherein the expression level of said marker is at least about 2 average log fold change more than the expression of said marker in native human beta cell.

19. The in vitro population pluripotent stem cell derived cells, according to any of the embodiment 15 or 16, wherein at least 95% of the beta like cells comprise STARD10, wherein the expression level of said marker is at least 1 average log fold change more than the expression of said marker in native human beta.

20. The in vitro population pluripotent stem cell derived cells, according to any of the embodiment 15 or 16, wherein at least 90% of the beta like cells comprise AMBP, wherein the expression level of said marker is at least 2 average log fold change more than the expression of said marker in native human beta.

21. The in vitro population pluripotent stem cell derived cells, according to embodiment 15, wherein at least 85% of the beta like cells comprise ST6GALNAC5, wherein the expression level of said marker is at least about 1 average log fold change more than the expression of said marker in native human beta.

22. The in vitro population pluripotent stem cell derived cells, according to any one of the embodiment 15 wherein at least 80% of the beta like cells comprise HMGCS1 wherein the expression level of said marker is at least about 1 average log fold change more than the expression of said marker in native human beta.

23. The in vitro population of pluripotent stem cell derived cells according to any of the preceding embodiments 4 to 22, wherein said cells further express G6PC2.

24. The in vitro population as claimed in any of the preceding embodiments, wherein the pluripotent stem cells are embryonic stem cells or induced pluripotent stem cells.

25. The in vitro population of pluripotent stem cell derived cells according to any one of the preceding embodiments 4 to 24, wherein said population comprising beta like cells maintains insulin expression and secretion after transplantation.

26. The in vitro population of pluripotent stem cell derived cells according to any of the preceding embodiments 4 to 25, wherein the population comprising beta like cells exhibit a glucose-stimulated insulin secretion (GSIS) response after transplantation.

27. The in vitro population of pluripotent stem cell derived cells according to any of the preceding embodiments 4 to 26, wherein one or more markers exhibit a positive correlation with average plasma c-peptide levels after transplantation

28. The in vitro population of pluripotent stem cell derived cells according to any of the preceding embodiments 4 to 27, wherein the population comprising beta like cells exhibit a lowering of blood glucose after transplantation.

29. The in vitro population of pluripotent stem cell derived cells according to any of the preceding embodiments 4 to 28 for use as a medicament.

30. The in vitro population of pluripotent stem cell derived cells according to any of the preceding embodiments 4 to 28 for use in the treatment of diabetes.

31. The in vitro population of pluripotent stem cell derived cells according to any of the preceding embodiments 4 to 28 for use in the treatment of type 1 diabetes.

32. A cell culture or composition, comprising the pluripotent stem cell derived cells according to any of the preceding embodiments 4 to 28 and a cell culture medium.

33. An implantable device comprising an in vitro population of pluripotent stem cell derived cells according to any of the preceding embodiments 4 to 28.

34. A method of treatment of type 1 diabetes, wherein the method comprises administering to a person in need thereof, an in vitro population of pluripotent stem cell derived cells according to any of the preceding embodiments 4 to 28.

35. A cryopreserved cell culture comprising in vitro population of pluripotent stem cell derived cells according to any of the preceding embodiments 4 to 28.

36. The cryopreserved population of pluripotent stem cell derived cells according embodiment 35, wherein at least 50%, 60%, 70%, 80% or 90% of the cell population is viable.

### List of Abbreviations

+ve: positive
AGN: AGN 193109
ALK5: Activin Receptor-like Kinases;
BC: Beta Cell
DE: Definitive Endoderm
EP: Endocrine Progenitor
GCG: Glucagon
hBS: human Blastocyst derived Stem
hBSC: human Blastocyst-derived Stem Cells
hES: human Embryonic Stem
hESC: human Embryonic Stem Cells
hiPSC: human induced Pluripotent Stem Cells
hPSC: human Pluripotent Stem Cells
LDN: LDN193189
PAX4: Paired Box 4
PE: Pancreatic Endoderm
RT: Room Temperature
SCID: Severe combined immunodeficiency
SYT13: Synaptotagmin 13
T3: Triiodothyronine

### Example 1

A Sub-Population of Stem Cell Derived Beta Like Cells Contribute to Insulin Secretion after Transplantation

Stem cell derived cells are heterogeneous based on gene expression, molecular properties, glucose sensing and insulin secretion. The aim of this experiment was to identify a sub-population of stem cell derived cells or beta like cells that contribute to insulin production after transplantation. To increase protocol related cell heterogeneity human embryonic stem cells (hESCs) were differentiated into endocrine cell clusters using 8 different modifications of a step wise differentiation protocol as provided below in Table 1. The hESCs were directed to Definitive endoderm (DE) followed by Pancreatic endoderm (PE), then Endocrine progenitors (EP) and finally insulin producing beta like cells were generated as described in patent publications WO/2012/175633, WO2014/033322, WO2015/028614, WO2017/144695 respectively (incorporated herein by reference in entirety).

TABLE 1
Eight (modified) differentiation protocols

| | Pancreatic | Endocrine | Beta cells |
|---|---|---|---|
| | endoderm (PE) | Progenitor (EP) | (BC) |
| Protocol | | | |
| Protocol 1 | No AGN | Alk5i/Nicotinamide, T3 | |
| Protocol 2 | AGN | Alk5i/Nicotinamide, T3 | |
| | (PE02-PE03) | | |
| Protocol 3 | AGN/LDN | Alk5i/Nicotinamide, T3 | |
| | (PE00-PE01) | Activin + Artemeter | |
| Protocol 4 | AGN/LDN | Alk5i/Nicotinamide, T3 | |
| | (PE00-PE01) | | |
| Protocol 5 | No AGN | Alk5i/Nicotinamide, No T3 | |
| Protocol 6 | AGN/LDN | Alk5i/Nicotinamide, T3 | No Alk5i |
| | (PE00-PE01) | | |
| Human islets | | | |
| Protocol 8 | AGN | No Alk5i/Nicotinamide, T3 | |
| | (PE02-PE03) | | |
| Protocol 9 | AGN | No Alk5i/Nicotinamide, T3 | No ALK5i |

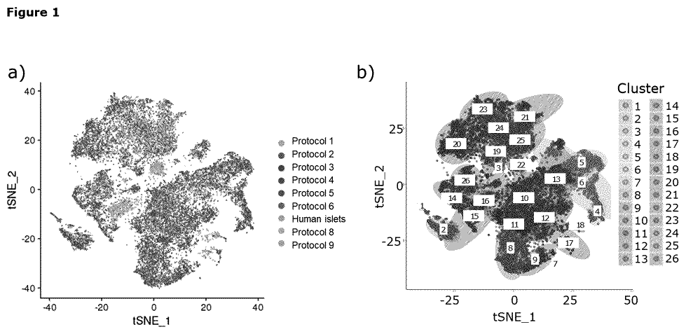

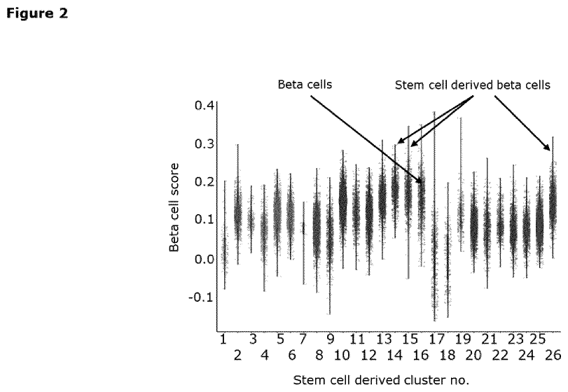

To identify sub-populations of stem cell derived cells or beta like cells that contributes to insulin expression and secretion after transplantation, cell clusters obtained from each of the 8 (modified) differentiation protocols were analyzed using single cell RNA sequencing prior to transplantation. The cells were dissociated into single cells using accutase (Stem Cell Technologies) and gene expression profiles for single cells were obtained for between 2000-3000 cells per protocol using RNA sequencing. Human islets were also included as a reference to native human beta cells. The gene expression data for all cells were integrated across all samples to make data comparable across conditions and dimension reduction and clustering was applied so that cells with a similar gene expression profile would group together independent of the protocol used to generate the cells. A total of 26 different cell populations were identified (FIG. 1b) and revealed that all protocols generated similar cell populations but in very different ratios (FIG. 1a). To identify those populations which mostly resembled the native human beta cells (cluster 16), each of the 26 identified clusters were compared to a native beta cell gene-set and clusters 14, 15 and 26 received a score similar to the human beta cell cluster (cluster 16), and hence were identified as the beta like cell populations that resembled native human beta cells the most (FIG. 2).

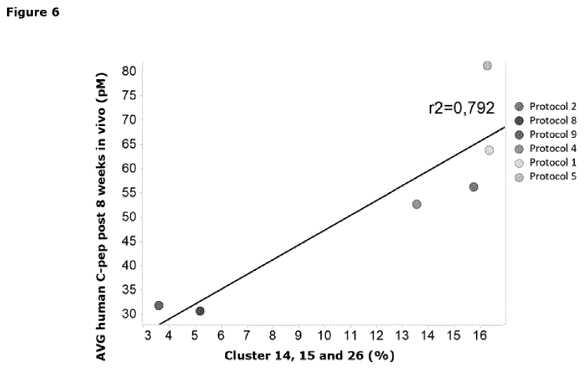

To determine insulin expression after transplantation in animals, a number of differentiated cell clusters (corresponding to 3000 plEQ) obtained with each of the 8 (modified) differentiation protocols were transplanted under the kidney capsule of immunocompromised SCID-beige mice in groups of 10 animals per protocol. As a measurement for insulin secretion from the engrafted human cells, human C-peptide levels were measured in serum samples 8 weeks after transplantation using human C-peptide ELISA (enzyme-linked immunoassay). By plotting the average circulating human C-peptide levels in the transplanted animals against the percentages of cells in clusters 14, 15 and 26 (out of the total cell number) a positive correlation was found between the percentage of cells in these clusters and insulin secretion in vivo (FIG. 6).

In conclusion, it was found that only a subpopulation of stem cell derived cells i.e. beta like cells resemble the native human beta cells and contribute to insulin secretion after transplantation. A list of genes that describe and identify this subpopulation or the beta like cells (cluster 14, 15 and 26) is provided in Tables 1, 2 and 3, see examples 2-4 provided below. Identification and characterisation of this population is essential for stem cell to beta cell differentiation protocol optimization and eventually for obtaining a beta cell therapy with high efficacy i.e. the ability of the cells to normalize blood glucose.

### Example 2 Conventional Beta Cell Markers do not Predict Insulin Secretion after Transplantation

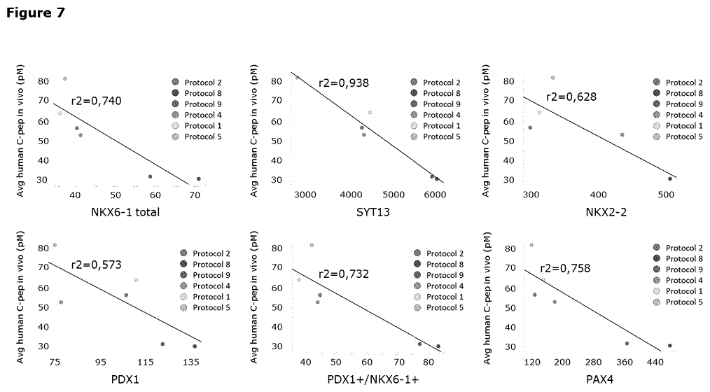

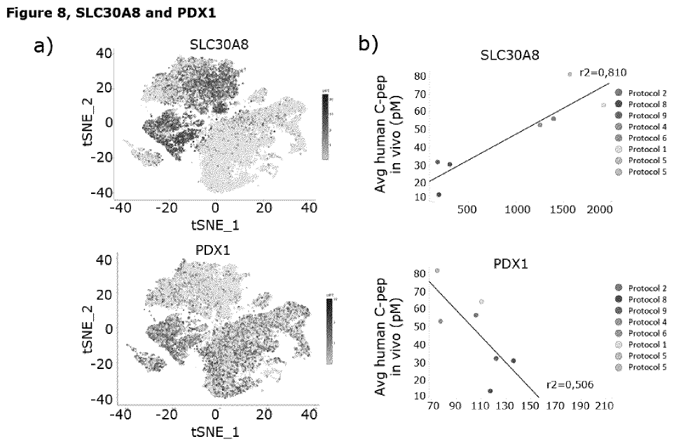

To show that conventional beta cell markers do not predict in vivo efficacy per se, we measured the expression of well-known beta cells markers such as NKX6.1, PDX1, PAX4, NKX2.2, SYT13 as well as co-expression of PDX1+/NKX6.1+ using either nanostring for detection of mRNA or flow cytometry for detection of protein in the cell populations obtained from the 8 modified protocols. We found that none of these markers correlated positively with the average c-peptide levels from the animals 8 weeks after transplantation (FIG. 7 and FIG. 8b, lower panel).

Through single cell sequence analysis, a few markers that previously have been associated with beta cells, including SLC30A8 were identified. These markers are listed below in Table 2.

TABLE 2
Table of genes enriched in the most beta like cell subset

| | | | | | medianUPT | medianUPT |
|---|---|---|---|---|---|---|
| Gene | | | | | (most beta | (all other |
| name | Gene | Avg_LogFC | P_val | P_val_adj | like cluster) | clusters) |
| SLC30A8 | ENSG00000164756 | 0.857934023 | 0 | 0 | 4.283072171 | 0 |
| DLK1 | ENSG00000185559 | 0.63358045 | 0 | 0 | 3.152261692 | 0 |
| MAFB | ENSG00000204103 | 0.543264842 | 0 | 0 | 4.672084243 | 2.188662727 |
| ISL1 | ENSG00000016082 | 0.468743458 | 0 | 0 | 2.643521175 | 0 |

To show how SLC30A8 is distinguished from another well-known beta cell marker, PDX1, in stem cell derived cells, we displayed their expression across all identified clusters (FIG. 8a). We found high expression of SLC30A8 in the most beta like cells (cluster 14, 15 and 26) as well as in the native beta cells, and some expression in clusters 24 and 25, which also express GCG. In contrast, although also expressed in the beta cells and most beta like cells (cluster 14, 15 and 26), PDX1 is also expressed in almost all other clusters. To show how SLC30A8 and PDX1 correlated to insulin secretion after transplantation we measured the levels of SLC30A8 and PDX1 mRNA transcript on nanostring in the 8 modified protocols and correlated this to average C-peptide level in vivo 8 weeks after transplantation. We found that SLC30A8 correlated positively (FIG. 8b, upper panel), whereas PDX1 correlated negatively (FIG. 8b, lower panel) to the circulating human C-peptide levels after transplantation. The expression of PDX1 in almost all cell clusters explains why this marker is negatively correlated to in vivo insulin secretion; although, PDX1 is expressed in the native beta cells and the most beta like cells from the stem cell cultures (cells in cluster 14, 15 and 26) the expression in the other clusters shows that this marker cannot identify the cells that will mature into fully functional beta cells after transplantation. As we show that certain classical beta cell markers do not per se predict insulin secretion after transplantation, we consider the application of the markers presented in earlier mentioned table 2 to be novel.

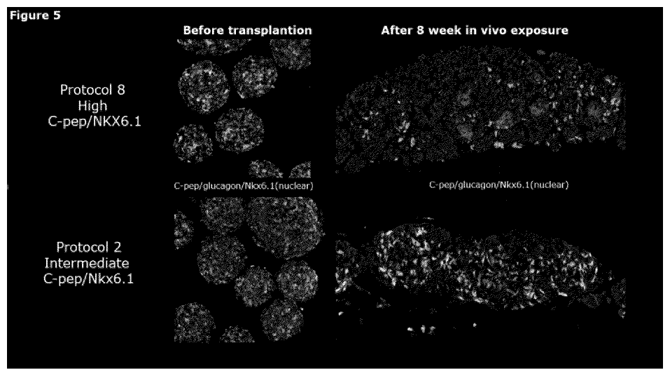

To further assess the cell composition before and after transplantation we did immunohistochemistry (IHC) on clusters before transplantation and on kidney section with engrafted human cells after transplantation. To identify alpha and beta cells, we used the conventional markers and stained for C-peptide, NKX6.1 and Glucagon. We found that from all 8 protocols a high percentage of the cells co-expressed C-pep/NKX6.1 before transplantation (FIG. 5, left panel), however only a subset of cells maintained this co-expression of C-pep/NKX6.1 after transplantation (FIG. 5, right panel).

In conclusion, we showed that most conventional beta cell markers do not predict insulin secretion after transplantation from in vitro stem cell derived endocrine cell cultures. The use of those conventional markers for cell screening or cell selection may lead to optimisation towards the undesired cell subpopulations that may look similar to beta cell before transplantation but changes their phenotype after exposure to an in vivo environment.

### Example 3

### Stem Cell Derived Beta Like Cells are Similar but not Identical to Native Beta Cells

To identify genes that are expressed in the stem derived beta like cells but not in native human beta cells the gene expression of these stem cell derived beta like cells was measured by single cell RNAseq and compared to the gene expression of native human beta cells.

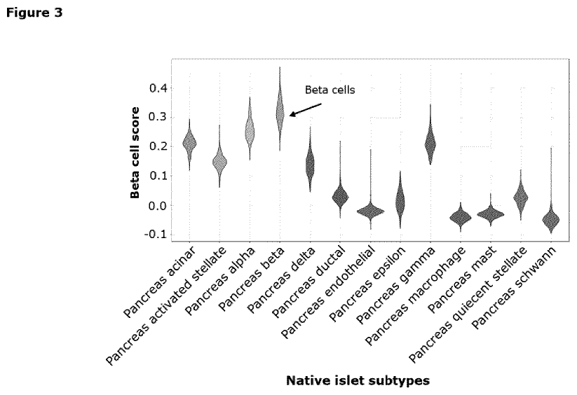

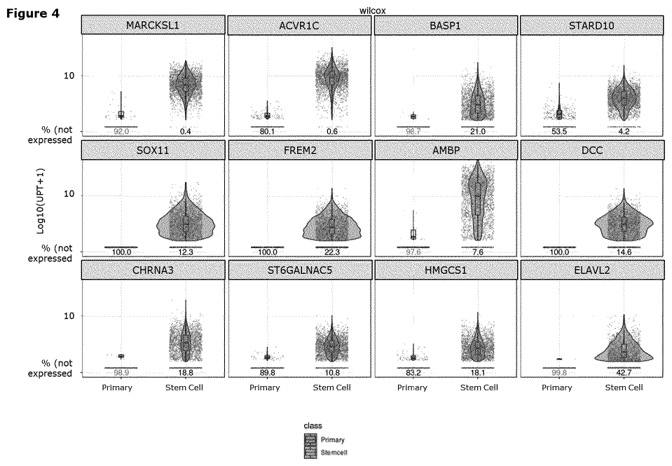

To obtain the gene expression of native human beta cells we pooled together single cell gene expression data from two human islet samples originating from two different human donors. To identify those cells within the islets that were native human beta cells, each cell from these samples were scored against gene sets of native islet subtype: alpha, beta, delta, epsilon, gamma, duct, acinar, endothelia, stellate, ductal, macrophages, mast and schwann cells (FIG. 3). The cells that scored highest for beta cells, were clustered together for comparison to cluster 14, 15 and 26. Comparing the two groups led to the identification of set of genes that are unique to the stem cell derived beta like cells (Table 3).

TABLE 3
Genes unique to Stem cell derived beta like cells.

| Gene | | | | | | MedianUPT |
|---|---|---|---|---|---|---|
| name | Gene | p_val | avg_logFC | pct. 1 | pct. 2 | Stemcell |
| ACVR1C | | | | | | |
| MARCKSL1 | ENSG00000175130 | 5.28E−251 | 1.899955 | 0.999 | 0.467 | 5.562079 |
| BASP1 | ENSG00000176788 | 8.71E−241 | 1.250194 | 0.985 | 0.69 | 1.1859583 |
| STARD10 | ENSG00000214530 | 9.62E−239 | 1.221227 | 0.992 | 0.513 | 2.4946988 |
| SOX11 | ENSG00000176887 | 2.21E−237 | 1.208628 | 0.98 | 0.639 | 1.5640478 |
| FREM2 | ENSG00000150893 | 1.40E−235 | 0.975562 | 0.966 | 0.013 | 1.054463 |
| AMBP | ENSG00000106927 | 1.05E−233 | 2.189005 | 0.98 | 0.75 | 7.4425528 |
| DCC | ENSG00000187323 | 6.98E−226 | 1.047687 | 0.972 | 0.929 | 1.5382249 |
| CHRNA3 | ENSG00000080644 | 5.78E−222 | 1.079882 | 0.957 | 0.597 | 1.5922722 |
| ST6GALNAC5 | ENSG00000117069 | 1.01E−219 | 0.842571 | 0.98 | 0.947 | 1.4145772 |
| HMGCS1 | ENSG00000112972 | 1.06E−219 | 0.839192 | 0.962 | 0.445 | 1.10011 |
| ELAVL2 | ENSG00000107105 | 8.65E−217 | 0.644576 | 0.908 | 0.219 | 0.4583582 |

In conclusion, we identified genes that distinguish the stem cell derived beta like cell from human native beta cells, showing that although similar these cells are not identical to their native counterpart.

### Example 4

ACVR1C is a Marker of Pancreatic Endocrine Cells Novel to Stem Cell Derived Beta Cell and its Expression Correlates with Insulin Secretion after Maturation In Vivo

ACVR1C is on the list of novel genes that is expressed in clusters 14, 15 and 26 (Table 4), the clusters identified among all clusters as resembling the most to native human beta cells.

TABLE 4
Table of novel genes enriched in clusters 14, 15 and 26

| | | | | | medianUPT | medianUPT |
|---|---|---|---|---|---|---|
| | | | | | (most beta | (all other |
| Gene name | Gene | avg_logFC | p_val | p_val_adj | like clusters) | clusters) |
| NEFM | ENSG00000104722 | 1.783094985 | 0 | 0 | 1.282791692 | 0 |
| PCP4 | ENSG00000183036 | 1.247749961 | 0 | 0 | 1.810064015 | 0 |
| PCDH7 | ENSG00000169851 | 1.021594162 | 0 | 0 | 1.337479533 | 0 |
| NEFL | ENSG00000277586 | 0.842066712 | 0 | 0 | 0 | 0 |
| PLAGL1 | ENSG00000118495 | 0.757018425 | 0 | 0 | 0.896942649 | 0 |
| EGFL7 | ENSG00000172889 | 0.6885571 | 0 | 0 | 0.468571243 | 0 |
| RAD21 | ENSG00000164754 | 0.65785573 | 0 | 0 | 2.717995272 | 1.331557923 |
| ACVR1C | ENSG00000123612 | 0.653054814 | 0 | 0 | 6.713394247 | 1.9251131 |
| RTN1 | ENSG00000139970 | 0.548424183 | 0 | 0 | 0.698104715 | 0 |
| PLXNA2 | ENSG00000076356 | 0.519268539 | 0 | 0 | 0.809031939 | 0 |
| LBH | ENSG00000213626 | 0.510902765 | 0 | 0 | 0 | 0 |

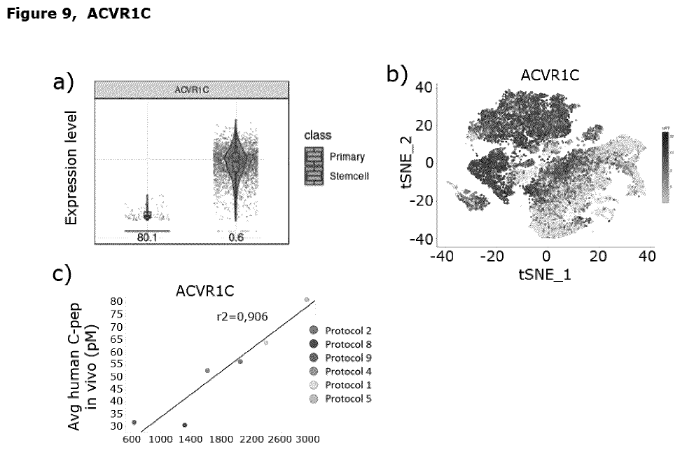

By using the method described in example 3, ACVR1C was identified as unique to stem cell derive beta like cells as it was expressed in more than 99% of the cells in cluster 14, and 26, and not expressed in 80% of the native human beta cells. The remaining 20% of the cells showed low ACVR1C expression (FIG. 9a). ACVR1C was highly expressed in clusters 14, 15 and 16 but also showed some expression in other clusters that map to other pancreatic endocrine subtypes, such as pancreatic alpha cells and pancreatic somatostatin cells. (FIG. 9b). To show that ACVR1C expression correlated positively to insulin secretion in vivo we measured the levels of ACVR1C mRNA transcript on nanostring and correlated this to average c-peptide level in vivo 8 weeks after transplantation (FIG. 9c).

In conclusion, we identified ACVR1C as a novel and unique marker of stem cell derived beta like cells that correlates to in vivo C-peptide levels 8 weeks after transplantation. C-peptide is equal to insulin. human C-peptide is measured as this can only originate from the transplanted cells and not from a potential externally given treatment with insulin. ACVR1C could hence be used to qualify and optimise protocol for stem cell derived beta like cells.

### Example 5

PCDH7 is an Extracellular and PCP4 is an Intracellular Marker that is Specific to the Most Beta Like Cell Populations

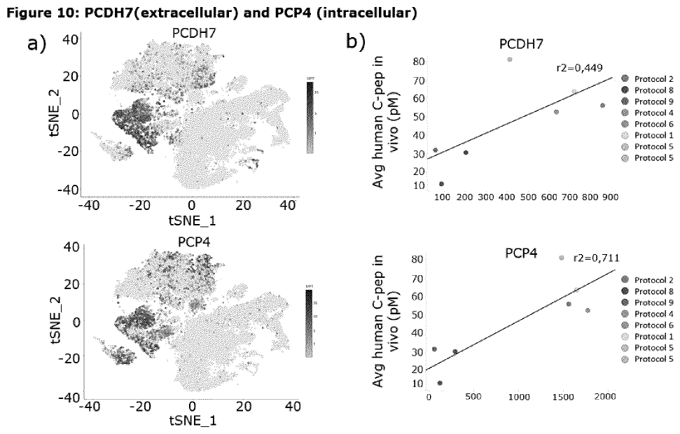

PCDH7 and PCP4 are both expressed in cell clusters 14, 15 and 26 (Table 4), the clusters in our single cell RNA sequencing data set identified among all clusters as resembling the most to native human beta cells. Compared to ACVR1C, PCDH7 and PCP4 show expression that is specific to the clusters 14, 15, and 26. No or very little expression of PCDH7 and PCP4 is found in any of the other identified clusters (FIG. 10a). The specificity of these markers makes them suitable for predicting insulin secretion after transplantation and elutes to their potential to be used to identify and quantify stem cell derived beta like cells for their use as quality markers. As for ACVR1C, we measured the levels of PCDH7 and PCP4 mRNA transcript on nanostring and correlated this to average circulating human c-peptide levels in mice in vivo 8 weeks after transplantation (FIG. 10b). We found that both markers correlated positively to insulin secretion in vivo after 8 weeks.

As PCDH7 is identified as an extracellular marker almost exclusively expressed in stem cell derived beta like cells, it can in addition be used to purify the stem cell derived beta cells from a heterogenous cell population. This can be used for further characterisation or to improve efficacy and safety of a final cell therapy product. In conclusion we identified PCDH7 and PCP4 as specific to the stem cell derived beta cells.

### Example 6

G6PC2 is a Marker that Correlates to In Vivo Insulin Production as Well as In Vitro Insulin Release

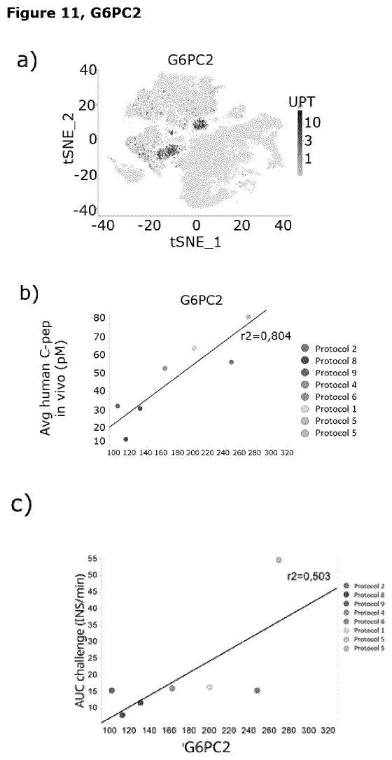

To identify markers that could be used for additional potency evaluation of the stem cell derived beta like cells, we measured the cells ability to secrete insulin in vitro for all 8 protocol modifications. Insulin secretion was measured using a perifusion system where the cells were first challenged with 10 mM glucose followed by a second challenge with 10 mM glucose+1000 nM Exendin4 followed by a third challenge by IBMX and Forskolin. The total amount of insulin secreted during the challenge was defined as the area under the perifusion curve and plotted against markers that positively correlated to in vivo insulin production. G6PC2 was found to be expressed in native human beta and alpha cells (cluster 16 and 22 respectively in single cell RNA sequencing data set) and in a subset of cell within the most beta like cells cluster 14, 15 and 26 (FIG. 11a). To show that G6PC2 expression correlated positively to insulin production in vivo we measured the levels of G6PC2 mRNA transcript on nanostring and correlated this to average c-peptide levels in vivo 8 weeks after transplantation (FIG. 11b). Finally, to validate its use as a potential potency marker for insulin secretion we compared the G6PC2 expression to the area under the curve from the in vitro insulin secretion test (FIG. 11c).

In conclusion, we identified GCPC2 as a marker expressed in a stem cell derived beta like cells which correlates to in vitro insulin secretion and hence is useful for measuring in vitro potency or in vitro differentiation of stem cells into beta cells.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1.-15. (canceled)

16. An in vitro population of beta like cells expressing NKX6.1 in combination with one or more markers selected from the group consisting of ACVR1C, MARCKSL1, BASP1, STARD10, AMBP, ST6GALNAC5, HMGCS1, SOX11, FREM2, and DCC;
wherein the expression level of each marker is at least about 1 average log fold change more than the expression level of the marker in a native human beta cell; and
wherein the cells are capable of maintaining insulin expression and secretion after in vivo transplantation.

17. The beta like cells according to claim 16, wherein the beta like cells express one or more markers selected from the group consisting of ACVR1C, MARCKSL1, BASP, STARD10, AMBP, ST6GALNAC5, and HMGCS1.

18. The beta like cells according to claim 16, wherein the beta like cells express one or more markers selected from the group consisting of SOX11, FREM2, and DCC.

19. The beta like cells according to claim 16, wherein the beta like cells further express G6PC2.

20. The beta like cells according to claim 16, wherein the beta like cells express ACVR1C, MARCKSL1, BASP, STARD10, AMBP, ST6GALNAC5, HMGCS1, SOX11, FREM2, and DCC.

21. The beta like cells according to claim 20, wherein the beta like cells further express G6PC2.

22. The beta like cells according to claim 16, wherein at least 95% of the beta like cells express ACVR1C.

23. The beta like cells according to claim 22, wherein the expression level of ACVR1C is at least about 2 average log fold change more than the expression of ACVR1C in a native human beta cell.

24. The beta like cells according to claim 16, wherein at least 95% of the beta like cells express MARCKSL1.

25. The beta like cells according to claim 24, wherein the expression level of MARCKSL1 is at least about 2 average log fold change more than the expression of MARCKSL1 in a native human beta cell.

26. The beta like cells according to claim 16, wherein at least 75% of the beta like cells express BASP1.

27. The beta like cells according to claim 16, wherein at least 95% of the beta like cells express STARD10.

28. The beta like cells according to claim 16, wherein at least 90% of the beta like cells express AMBP.

29. The beta like cells according to claim 28, wherein the expression level of AMBP is at least about 2 average log fold change more than the expression of AMBP in a native human beta cell.

30. The beta like cells according to claim 16, wherein at least 85% of the beta like cells express ST6GALNAC5.

31. The beta like cells according to claim 16, wherein at least 80% of the beta like cells express HMGCS1.

32. The beta like cells according to claim 16, wherein at least 85% of the beta like cells express SOX11.

33. The beta like cells according to claim 16, wherein at least 75% of the beta like cells comprise FREM2.

34. The beta like cells according to claim 16, wherein at least 85% of the beta like cells comprise DCC.

35. The beta like cells according to claim 16, wherein at least 95% of the beta like cells express ACVR1C, wherein at least 95% of the beta like cells express MARCKSL1, wherein at least 75% of the beta like cells express BASP1, wherein at least 95% of the beta like cells express STARD10, wherein at least 90% of the beta like cells express AMBP, wherein at least 85% of the beta like cells express ST6GALNAC5, wherein at least 80% of the beta like cells express HMGCS1, wherein at least 85% of the beta like cells express SOX11, wherein at least 75% of the beta like cells comprise FREM2, and wherein at least 85% of the beta like cells comprise DCC.

36. The beta like cells according to claim 16, wherein at least 80% of the beta like cells express ACVR1C, MARCKSL1, STARD10, AMBP, ST6GALNAC5, and HMGCS1.

37. The beta like cells according to claim 16, wherein at least 90% of the beta like cells express ACVR1C, MARCKSL1, STARD10, and AMBP.

38. The beta like cells according to claim 16, wherein at least 55% of the beta like cells express SOX11, FREM2, and DCC.

39. The beta like cells according to claim 16, wherein at least 75% of the beta like cells express SOX11, FREM2, and DCC.

40. A method for screening beta like cells in an in vitro population of pluripotent stem cell derived cells, comprising identifying beta like cells expressing NKX6.1 in combination with one or more markers selected from the group consisting of ACVR1C, MARCKSL1, BASP1, STARD10, AMBP, ST6GALNAC5, HMGCS1, SOX11, FREM2, and DCC;
wherein the expression level of each marker is at least about 1 average log fold change more than the expression level of the marker in a native human beta cell; and
wherein the cells are capable of maintaining insulin expression and secretion after in vivo transplantation.

41. The method according to claim 40, wherein the beta like cells express one or more markers selected from the group consisting of ACVR1C, MARCKSL1, BASP, STARD10, AMBP, ST6GALNAC5, and HMGCS1.

42. The beta like cells according to claim 40, wherein the beta like cells express one or more markers selected from the group consisting of SOX11, FREM2, and DCC.

43. The beta like cells according to claim 40, wherein the beta like cells further express G6PC2.

44. The beta like cells according to claim 40, wherein the beta like cells express ACVR1C, MARCKSL1, BASP, STARD10, AMBP, ST6GALNAC5, HMGCS1, SOX11, FREM2, and DCC.

45. The beta like cells according to claim 44, wherein the beta like cells further express G6PC2.

46. The method according to claim 40, wherein at least 95% of the beta like cells express ACVR1C.

47. The method according to claim 46, wherein the expression level of ACVR1C is at least about 2 average log fold change more than the expression of ACVR1C in a native human beta cell.

48. The method according to claim 40, wherein at least 95% of the beta like cells express MARCKSL1.

49. The method according to claim 48, wherein the expression level of MARCKSL1 is at least about 2 average log fold change more than the expression of MARCKSL1 in a native human beta cell.

50. The method according to claim 40, wherein at least 75% of the beta like cells express BASP1.

51. The method according to claim 40, wherein at least 95% of the beta like cells express STARD10.

52. The method according to claim 40, wherein at least 90% of the beta like cells express AMBP.

53. The method according to claim 52, wherein the expression level of AMBP is at least about 2 average log fold change more than the expression of AMBP in a native human beta cell.

54. The method according to claim 40, wherein at least 85% of the beta like cells express ST6GALNAC5.

55. The method according to claim 40, wherein at least 80% of the beta like cells express HMGCS1.

56. The method according to claim 40, wherein at least 85% of the beta like cells express SOX11.

57. The method according to claim 40, wherein at least 75% of the beta like cells comprise FREM2.

58. The method according to claim 40, wherein at least 85% of the beta like cells comprise DCC.

59. The method according to claim 40, wherein at least 95% of the beta like cells express ACVR1C, wherein at least 95% of the beta like cells express MARCKSL1, wherein at least 75% of the beta like cells express BASP1, wherein at least 95% of the beta like cells express STARD10, wherein at least 90% of the beta like cells express AMBP, wherein at least 85% of the beta like cells express ST6GALNAC5, wherein at least 80% of the beta like cells express HMGCS1, wherein at least 85% of the beta like cells express SOX11, wherein at least 75% of the beta like cells comprise FREM2, and wherein at least 85% of the beta like cells comprise DCC.

60. The method according to claim 40, wherein at least 80% of the beta like cells express ACVR1C, MARCKSL1, STARD10, AMBP, ST6GALNAC5, and HMGCS1.

61. The method according to claim 40, wherein at least 90% of the beta like cells express ACVR1C, MARCKSL1, STARD10, and AMBP.

62. The method according to claim 40, wherein at least 55% of the beta like cells express SOX11, FREM2, and DCC.

63. The method according to claim 40, wherein at least 75% of the beta like cells express SOX11, FREM2, and DCC.
